(19) United States
(12) Patent Application Publication
(10) Pub. No.: US 2026/0026787 A1
(43) Pub. Date: Jan. 29, 2026

(54) ULTRASONIC DIAGNOSTIC APPARATUS

(71) Applicant: CANON MEDICAL SYSTEMS CORPORATION, Otawara-shi (JP)

(72) Inventor: Minori OHASHI, Otawara (JP)

(73) Assignee: CANON MEDICAL SYSTEMS CORPORATION, Otawara-shi (JP)

(21) Appl. No.: 19/274,728

(22) Filed: Jul. 21, 2025

(30) Foreign Application Priority Data
Jul. 25, 2024 (JP) 2024-120649
Jul. 16, 2025 (JP) 2025-119853

(51) Int. Cl. A61B 8/00; A61B 8/06; G01S 15/89

(52) U.S. Cl. CPC A61B 8/5246; A61B 8/06; A61B 8/4254; A61B 8/463; A61B 8/488; A61B 8/5207; G01S 15/8979

(57) ABSTRACT

An ultrasonic diagnostic apparatus according to an embodiment includes an acquisition unit configured to acquire a two-dimensional ultrasonic image generated based on a signal received by an ultrasonic probe and first measurement position information related to a measurement position for measurement of blood flow information, the measurement position being set in the two-dimensional ultrasonic image, a calculation unit configured to calculate a matching degree between the two-dimensional ultrasonic image and the first measurement position information, and a reference image having a measurement position set therein and second measurement position information related to the measurement position set in the reference image, and a transition control unit configured to control, based on a calculation result of a calculation unit, a transition to a Doppler mode for the measurement of the blood flow information in a subject.

### CROSS REFERENCE TO RELATED APPLICATIONS

This application is based upon and claims the benefit of priority from the prior Japanese Patent Applications No. 2024-120649, filed on Jul. 25, 2024, and No. 2025-119853, filed on Jul. 16, 2025, the entire contents of which are incorporated herein by reference.

### FIELD

Embodiments described in the present specification and drawings relate to an ultrasonic diagnostic apparatus.

### BACKGROUND

Conventionally, in an ultrasonic diagnostic apparatus, Doppler spectrum (Doppler waveform), which is an example of blood flow information, is displayed using Doppler information (Doppler signal) extracted from a reflected wave of an ultrasonic wave. The Doppler waveform is a waveform obtained by plotting the blood flow velocity at the measurement position set by a user as an observation site along the time series.

For example, in a PWD mode for measurement of a Doppler waveform by a pulsed wave Doppler (PWD) method, a user manually arranges a marker indicating a measurement position at a specific site in a blood vessel in accordance with traveling of the blood vessel depicted in a two-dimensional ultrasonic image (two-dimensional B-mode image or two-dimensional color Doppler image), and the user presses a PWD button for transitioning to the PWD mode to transition to the PWD mode, whereby the Doppler waveform, which is an example of blood flow information at the measurement position, is displayed live on a display of the ultrasonic diagnostic apparatus. In a case where the mode transitions to the PWD mode in this manner, the user needs to operate the ultrasonic probe with one hand to draw an appropriate two-dimensional ultrasonic image on the display, and operate an input device with the other hand to execute the transition to the PWD mode by pressing the PWD button while placing a marker at a specific site in the blood vessel on the visualized two-dimensional ultrasonic image. That is, the user needs to simultaneously perform the operation of the ultrasonic probe for drawing the two-dimensional ultrasonic image and the input operation for transitioning to the PWD mode while arranging the marker at a specific site in the blood vessel on the two-dimensional ultrasonic image, and the user's operation is complicated.

Such a problem occurs not only in the PWD mode but also in a Doppler mode such as a continuous wave Doppler (CWD) mode for measurement of a Doppler waveform by a CWD method. Therefore, it is desirable to allow the user to focus on drawing the two-dimensional ultrasonic image while assisting the user's operation from transitioning to the Doppler mode.

### BRIEF DESCRIPTION OF THE DRAWINGS

FIG. 1 is a block diagram illustrating an example of a configuration of an ultrasonic diagnostic apparatus according to a first embodiment;

FIG. 2 is a diagram illustrating an example of a target image and second measurement position information according to the first embodiment;

FIG. 3 is a diagram illustrating an example of a two-dimensional ultrasonic image and a Doppler waveform displayed on a display in the ultrasonic diagnostic apparatus according to the first embodiment;

FIG. 4 is a flowchart for description of mode transition processing executed in the ultrasonic diagnostic apparatus according to the first embodiment;

FIG. 5 is a block diagram illustrating an example of a configuration of an ultrasonic diagnostic apparatus according to a third embodiment;

FIG. 6 is a flowchart for description of mode transition processing executed in the ultrasonic diagnostic apparatus according to the third embodiment;

FIG. 7 is a block diagram illustrating an example of a configuration of an ultrasonic diagnostic apparatus according to a fourth embodiment;

FIG. 8 is a flowchart for description of mode transition processing executed in the ultrasonic diagnostic apparatus according to the fourth embodiment;

FIG. 9 is a diagram illustrating an example of a two-dimensional ultrasonic image and a Doppler waveform displayed on a display in the ultrasonic diagnostic apparatus according to the fourth embodiment;

FIG. 10 is a block diagram illustrating an example of a configuration of an ultrasonic diagnostic apparatus according to a fifth embodiment;

FIG. 11 is a flowchart for description of mode transition processing executed in the ultrasonic diagnostic apparatus according to the fifth embodiment;

FIG. 12 is a diagram illustrating an example of a two-dimensional ultrasonic image, a Doppler waveform, and a target image displayed on a display in an ultrasonic diagnostic apparatus according to a sixth embodiment;

FIG. 13 is a block diagram illustrating an example of a configuration of an ultrasonic diagnostic apparatus according to a seventh embodiment;

FIG. 14 is a flowchart for description of mode transition processing executed in the ultrasonic diagnostic apparatus according to the seventh embodiment;

FIG. 15 is a diagram illustrating an example of a step in which an acquisition function acquires a two-dimensional B-mode image and a marker, which is an example of second identification information, in step S11 of mode transition processing executed in an ultrasonic diagnostic apparatus according to an eighth embodiment; and

FIG. 16 is a diagram illustrating an example of a step in which a calculation function calculates a matching degree between a two-dimensional ultrasonic image and a measurement position thereof, and a target image and a measurement position thereof in step S13 of the mode transition processing executed in the ultrasonic diagnostic apparatus according to the eighth embodiment.

### DETAILED DESCRIPTION

Hereinafter, respective embodiments of the ultrasonic diagnostic apparatus will be described with reference to the accompanying drawings. In the embodiments below, the same reference signs are given for identical components in terms of configuration and function, and duplicate description is omitted.

### First Embodiment

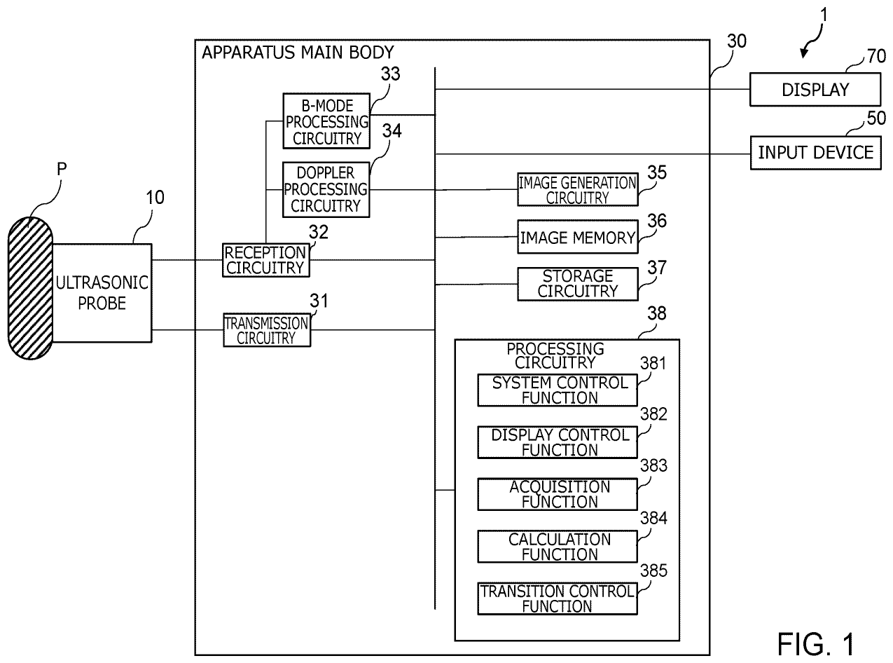

FIG. 1 is a block diagram showing an example of a configuration of an ultrasonic diagnostic apparatus according to a first embodiment. As illustrated in FIG. 1, an ultrasonic diagnostic apparatus 1 includes an ultrasonic probe 10, an apparatus main body 30, an input device 50, and a display 70.

The ultrasonic probe 10 includes, for example, a plurality of piezoelectric vibrators. The plurality of piezoelectric vibrators generate an ultrasonic wave based on a drive signal supplied from a transmission circuitry 31 included in the apparatus main body 30 to be described later. In addition, the ultrasonic probe 10 receives a reflected wave from a subject P and converts the reflected wave into an electrical signal. In addition, the ultrasonic probe 10 includes, for example, a matching layer provided on the piezoelectric vibrator, a backing material for preventing propagation of an ultrasonic wave rearwards from the piezoelectric transducer and the like.

When an ultrasonic wave is transmitted from the ultrasonic probe 10 to the subject P, the transmitted ultrasonic wave is reflected one after another on a discontinuous surface of an acoustic impedance in a body tissue of the subject P, and is received as the reflected wave signal by the plurality of piezoelectric vibrators included in the ultrasonic probe 10. The amplitude of the received reflected wave signal depends on the difference in acoustic impedance at the discontinuous surface at which ultrasonic waves are reflected. Note that a reflected wave signal in a case where transmitted ultrasonic pulses are reflected by a moving blood flow or a surface of a heart wall or the like receives a frequency shift depending on a velocity component with respect to the direction of ultrasonic transmission of a moving body, due to the Doppler effect.

The ultrasonic probe 10 is detachably connected to the apparatus main body 30. In a case where scanning a two-dimensional region in the subject P (two-dimensional scanning) is performed, the user connects, for example, a 1D array probe in which a plurality of piezoelectric vibrators are arranged in a line to the apparatus main body 30 as the ultrasonic probe 10. The 1D array probe is a linear type ultrasonic probe, a convex type ultrasonic probe, a sector type ultrasonic probe, or the like. In a case where scanning a three-dimensional region in the subject P (three-dimensional scanning) is performed, the user connects, for example, a mechanical 4D probe or a 2D array probe as the ultrasonic probe 10 to the apparatus main body 30. The mechanical 4D probe can perform two-dimensional scanning using a plurality of piezoelectric vibrators arranged in a line like the 1D array probe, and can perform three-dimensional scanning by swinging the plurality of piezoelectric vibrators at a predetermined angle (a swing angle). In addition, a 2D array probe can perform three-dimensional scanning by a plurality of piezoelectric vibrators arranged in a matrix, and can perform two-dimensional scanning by focusing and transmitting ultrasonic waves. Note that the 2D array probe can simultaneously perform two-dimensional scanning of a plurality of cross sections.

In addition, as will be described later, the ultrasonic diagnostic apparatus 1 according to the present embodiment measures a Doppler waveform by a pulsed wave Doppler (PWD) method or a continuous wave Doppler (CWD) method. Therefore, in the present embodiment, the ultrasonic probe 10 connected to the apparatus main body 30 is an ultrasonic probe of capable executing ultrasonic wave transmission/reception for capturing a two-dimensional ultrasonic image and ultrasonic wave transmission/reception for measuring a Doppler waveform by a PWD method or a CWD method.

The apparatus main body 30 generates an ultrasonic image based on a signal from the ultrasonic probe 10. Specifically, the apparatus main body 30 can generate a two-dimensional ultrasonic image based on a reflected wave signal corresponding to a two-dimensional region of the subject P received by the ultrasonic probe 10. In addition, the apparatus main body 30 can generate a three-dimensional ultrasonic image based on a reflected wave signal corresponding to a three-dimensional region of the subject P received by the ultrasonic probe 10. As illustrated in FIG. 1, the apparatus main body 30 includes the transmission circuitry 31, a reception circuitry 32, a B-mode processing circuitry 33, a Doppler processing circuitry 34, an image generation circuitry 35, an image memory 36, a storage circuitry 37, and a processing circuitry 38.

The transmission circuitry 31 includes a pulse generator, a transmission delay unit, a pulser, and the like, and supplies a drive signal to the ultrasonic probe 10. The pulse generator repeatedly generates rate pulses for forming transmission ultrasonic waves at a predetermined rate frequency. Further, the transmission delay unit focuses ultrasonic waves generated from the ultrasonic probe 10 into a beam shape, and gives, to each rate pulse generated by the pulse generator, a delay time for each piezoelectric vibrator necessary to determine transmission directivity. Further, the pulser applies a drive signal (drive pulse) to the ultrasonic probe 10 at a timing based on the rate pulse. That is, the transmission delay unit arbitrarily adjusts the transmission direction of the ultrasonic wave transmitted from the piezoelectric vibrator surface by changing the delay time given to each rate pulse.

Note that the transmission circuitry 31 has a function of instantaneously changing a transmission frequency, a transmission driving voltage, and the like in order to execute a predetermined scan sequence based on an instruction from the processing circuitry 38 to be described later. In particular, the transmission driving voltage is changed by a linear amplifier type transmission circuitry capable of instantaneously switching the value thereof or a mechanism for electrically switching a plurality of power supply units.

The reception circuitry 32 includes a preamplifier, an analog/digital (A/D) converter, a reception delay unit, an adder, and the like, and performs various types of processing on the reflected wave signal received by the ultrasonic probe 10, thereby generating reflected wave data. The preamplifier amplifies the reflected wave signal for each channel and performs gain adjustment (gain correction). The A/D converter A/D converts the gain-corrected reflected wave signal into a digital signal by A/D converting the gain-corrected reflected wave signal. The reception delay unit gives a delay time necessary to determine reception directivity. The adder performs processing of adding reflected wave signals processed by the reception delay unit, and generates reflected wave data. Then, the adder outputs the generated reflected wave data to the B-mode processing circuitry 33 and the Doppler processing circuitry 34.

In a case where the subject P is two-dimensionally scanned, the transmission circuitry 31 causes the ultrasonic probe 10 to transmit a two-dimensional ultrasonic beam. Then, the reception circuitry 32 generates two-dimensional reflected wave data from a two-dimensional reflected wave signal received by the ultrasonic probe 10. In addition, in a case where the subject P is three-dimensionally scanned, the transmission circuitry 31 according to the present embodiment causes the ultrasonic probe 10 to transmit a three-dimensional ultrasonic beam. Then, the reception circuitry 32 generates three-dimensional reflected wave data from a three-dimensional reflected wave signal received by the ultrasonic probe 10.

The B-mode processing circuitry 33 receives the reflected wave data from reception circuitry 32, performs logarithmic amplification, envelope detection processing, and the like, and generates data (B-mode data) in which signal intensity is expressed by brightness of luminance.

The Doppler processing circuitry 34 performs frequency analysis on velocity information from the reflected wave data received from the reception circuitry 32, extracts a blood flow, a tissue, and a contrast medium echo component by the Doppler effect, and generates data (Doppler data) obtained by extracting moving body information such as velocity, dispersion, and power for multiple points. Here, the moving body is, for example, a blood flow, a tissue of an organ that periodically moves, such as a heart wall, or a contrast medium.

The B-mode processing circuitry 33 and the Doppler processing circuitry 34 can process both two-dimensional reflected wave data and three-dimensional reflected wave data. That is, the B-mode processing circuitry 33 generates two-dimensional B-mode data from two-dimensional reflected wave data, and generates three-dimensional B-mode data from three-dimensional reflected wave data. In addition, the Doppler processing circuitry 34 generates two-dimensional Doppler data from two-dimensional reflected wave data and generates three-dimensional Doppler data from three-dimensional reflected wave data.

The image generation circuitry 35 generates an ultrasonic image expressed in a predetermined luminance range based on a signal received by the ultrasonic probe 10. For example, the image generation circuitry 35 generates, as the ultrasonic image, a two-dimensional B-mode image in which the intensity of the reflected wave is represented by luminance from the two-dimensional B-mode data generated by the B-mode processing circuitry 33. In addition, the image generation circuitry 35 generates, as an ultrasonic image, an average speed image, a distributed image, a power image, or a two-dimensional color Doppler image as a combination image thereof, representing the moving body information from the two-dimensional Doppler data generated by the Doppler processing circuitry 34.

Here, the image generation circuitry 35 generally converts (scan converts) a scanning line signal sequence of ultrasonic scanning into a scanning line signal sequence of a video format represented by a television or the like, and generates a display ultrasonic image. For example, the image generation circuitry 35 generates the display ultrasonic image by performing coordinate conversion according to a scanning mode of an ultrasonic wave by the ultrasonic probe 10. In addition, for example, the image generation circuitry 35 performs, as various types of image processing other than the scan conversion, image processing (smoothing processing) of regenerating an average value image of luminance by using a plurality of image frames after scan conversion, image processing (edge enhancement processing) using a differential filter in the image, and the like.

That is, the B-mode data and the Doppler data are data before scan conversion processing, and the data generated by the image generation circuitry 35 is a display ultrasonic image after the scan conversion processing. Note that the B-mode data and the Doppler data are also referred to as raw data. The image generation circuitry 35 generates the two-dimensional B-mode image or the two-dimensional color Doppler image, which is a two-dimensional ultrasonic image, from the two-dimensional B-mode data or the two-dimensional Doppler data, which is raw data. Furthermore, the image generation circuitry 35 can also generate, for example, a superimposed image in which a color Doppler image is superimposed on the two-dimensional B-mode image.

Furthermore, for example, the image generation circuitry 35 generates an M-mode image from the time-series data of the B-mode data on one scanning line generated by the B-mode processing circuitry 33. In addition, the image generation circuitry 35 generates a Doppler waveform obtained by plotting a blood flow and tissue velocity information along a time series from the Doppler data generated by the Doppler processing circuitry 34. This Doppler waveform is an example of blood flow information.

Furthermore, the image generation circuitry 35 can also generate a three-dimensional B-mode image by performing coordinate conversion on the three-dimensional B-mode data generated by the B-mode processing circuitry 33. Furthermore, the image generation circuitry 35 can also generate a three-dimensional color Doppler image by performing coordinate conversion on the three-dimensional Doppler data generated by the Doppler processing circuitry 34.

The image memory 36 is a memory that stores various images generated by the image generation circuitry 35. The image memory 36 also stores data generated by the B-mode processing circuitry 33 and the Doppler processing circuitry 34. The B-mode data and the Doppler data stored in the image memory 36 can be called by an operator after diagnosis, for example, and become a display ultrasonic image via the image generation circuitry 35. The image memory 36 also stores the reflected wave data output from the reception circuitry 32. For example, the image memory 36 is realized by a random access memory (RAM), a semiconductor memory element such as a flash memory, a hard disk, or an optical disk.

The storage circuitry 37 stores various types of data such as a control program for performing ultrasonic wave transmission/reception, image processing, and display processing, diagnostic information (for example, patient ID, doctor's finding, and the like), a diagnostic protocol, and various body marks. In addition, the storage circuitry 37 stores a threshold value related to a matching degree to be described later. In addition, the storage circuitry 37 is also used to store data stored in the image memory 36, as necessary. For example, the storage circuitry 37 is realized by a semiconductor memory element such as a flash memory, a hard disk, or an optical disk.

In addition, the storage circuitry 37 stores a target image. This target image corresponds to a reference image according to the present embodiment. Therefore, the storage circuitry 37 corresponds to a storage unit according to the present embodiment. Here, the target image is an ultrasonic image used for comparison with the two-dimensional ultrasonic image. In this target image, a measurement position for measuring blood flow information is set. Specifically, for example, a marker indicating a measurement position for measuring a Doppler waveform, which is an example of blood flow information, is set in the target image. The marker indicating the measurement position is also referred to as a sample volume or a sample gate. The marker indicating the measurement position in the target image is an example of second measurement position information, which is information related to the measurement position set in the reference image. The target image may be stored in the image memory 36. In this case, the image memory 36 corresponds to the storage unit according to the present embodiment.

The target image according to the present embodiment is, for example, an ultrasonic image in which a measurement position is set in a frozen state of a live display of the two-dimensional B-mode image. Specifically, in the ultrasonic inspection on the subject P, before the Doppler waveform is measured, when a user can draw a cross section (ultrasonic image) in B mode to be described later, and when the user is able to draw an intended cross section (ultrasonic image), this target image is an ultrasonic image in which a measurement position is set by freezing the live display of the two-dimensional B-mode image being displayed live on the display 70, and setting a marker indicating the measurement position on the frozen two-dimensional B-mode image displayed on the display 70 by the user. That is, the storage circuitry 37 stores, as a target image TA1, the frozen two-dimensional B-mode image displayed on the display 70, which is the ultrasonic image in which the measurement position is set. In addition, the storage circuitry 37 stores a marker indicating the measurement position set on the two-dimensional B-mode image as second measurement position information together with the frozen two-dimensional B-mode image displayed on the display 70.

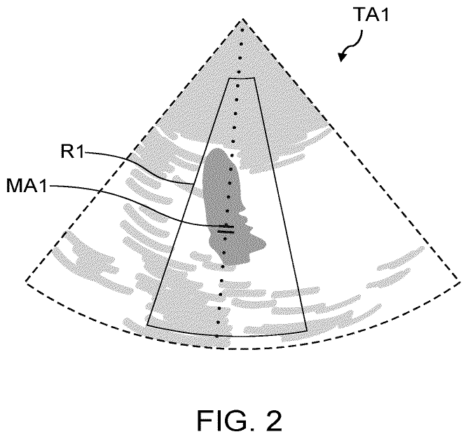

FIG. 2 is a diagram illustrating an example of the target image and the second measurement position information according to the first embodiment. As illustrated in FIG. 2, the target image TA1 is a two-dimensional B-mode image, and a marker MA1 indicating the measurement position in the target image TA1 is set on the target image TA1 as the second measurement position information. Furthermore, in the example illustrated in FIG. 2, a region of interest R1 for synthesizing a two-dimensional color Doppler image is set in the target image TA1. Note that the region of interest R1 may not be set in the target image TA1.

The processing circuitry 38 executes various types of data processing. The processing circuitry 38 includes a system control function 381, a display control function 382, an acquisition function 383, a calculation function 384, and a transition control function 385. Here, for example, each processing function of the system control function 381, the display control function 382, the acquisition function 383, the calculation function 384, and the transition control function 385, which are components of the processing circuitry 38 illustrated in FIG. 1, is recorded in the storage circuitry 37 in the form of a program executable by a computer. The processing circuitry 38 reads each program from the storage circuitry 37 and executes each read program to implement a function corresponding to each program. In other words, the processing circuitry 38 in a state of reading each program has each function illustrated in the processing circuitry 38 of FIG. 1. The processing circuitry 38 is realized by, for example, a processor. The system control function 381 is an example of a control unit. The display control function 382 is an example of a display control unit. The acquisition function 383 is an example of an acquisition unit. The calculation function 384 is an example of a calculation unit. The transition control function 385 is an example of a transition control unit.

The system control function 381 is a function of integrally controlling the entire operation of the ultrasonic diagnostic apparatus 1 in the system control function 381. For example, the system control function 381 controls the transmission circuitry 31 and the reception circuitry 32 based on parameters related to transmission and reception of ultrasonic waves according to various modes. The various modes include, for example, a B mode, a color Doppler mode, a PWD mode, a CWD mode, and the like. Note that the PWD mode is also referred to as a PW mode, and the CWD mode is also referred to as a CW mode. In the following description, the PWD mode and the CWD mode will be collectively referred to as a Doppler mode.

The B mode is a mode for generating a B mode image by B mode scanning. The color Doppler mode is a mode for generating a color Doppler image in which a color is allocated to blood flow information measured using, for example, a pulse wave by color Doppler mode scanning. The color Doppler mode scanning includes B mode scanning. Then, in the color Doppler mode, for example, both the B mode image and the color Doppler image are generated, and the color Doppler image is superimposed and displayed on the B mode image.

The PWD mode is a mode for measuring a Doppler waveform related to a specific measurement site by a PWD mode scan (PWD type scan) for transmitting a pulse wave to a scanning line and receiving a reflected wave. In the PWD mode, it is common for the ultrasonic probe 10 to perform the PWD mode scan on one scanning line, but it is also possible to perform the PWD mode scan on multiple scanning lines. In this case, the ultrasonic probe 10 sequentially transmits pulse waves to the plurality of scanning lines and receives reflected waves. In the PWD mode, only the Doppler waveform is updated in order to observe the blood flow with high image quality. In this case, the B-mode scan cannot be used together.

The CWD mode is a mode for measuring a Doppler waveform on one scanning line by CWD mode scanning (scanning of a CWD system) for receiving a reflected wave while transmitting a continuous wave. In the CWD mode, it is necessary to continuously apply the continuous wave to a target, and thus, the B-mode scan cannot be used together. Hereinafter, the present embodiment will be described with an example in which the Doppler mode is the PWD mode and the Doppler waveform is measured by the PWD method.

The display control function 382 is a function of controlling the display 70 so as to display various ultrasonic images, Doppler waveforms, and the like generated by the image generation circuitry 35. For example, the display control function 382 controls the display 70 to display the B-mode image generated by the image generation circuitry 35, the color Doppler image, or an image including both of them, and the Doppler waveform. Specifically, the display control function 382 displays the B-mode image, the color Doppler image, or the image including both of them live on the display 70, or displays the Doppler waveform live (in real time) on the display 70. In addition, the display control function 382 controls the display 70 to cause the two-dimensional ultrasonic image to be displayed live and to freeze the live display of the Doppler waveform, or controls the display 70 to freeze the live display of the two-dimensional ultrasonic image and to cause the Doppler waveform to be displayed live.

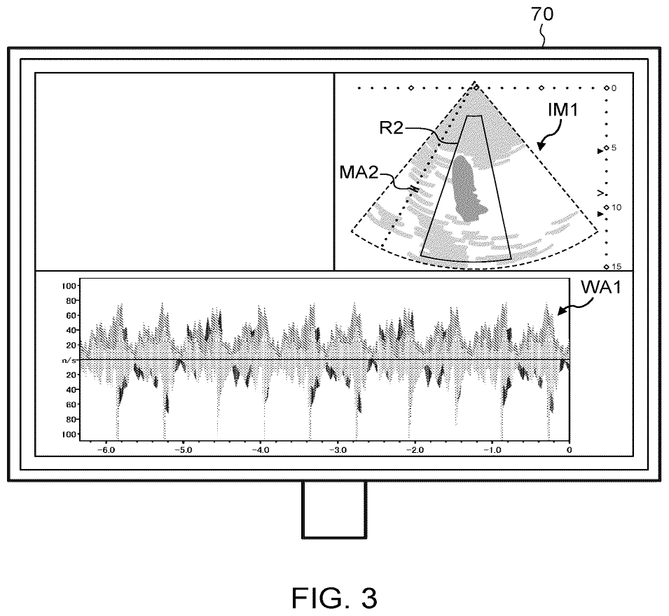

FIG. 3 is a diagram illustrating an example of the two-dimensional ultrasonic image and the Doppler waveform displayed on the display 70 in the ultrasonic diagnostic apparatus 1 according to the first embodiment. As illustrated in FIG. 3, the display control function 382 displays a two-dimensional B-mode image IM1 as a two-dimensional ultrasonic image and a Doppler waveform WA1 on the display 70. In addition, the display control function 382 displays a marker MA2 indicating a measurement position for measuring blood flow information on the two-dimensional B-mode image IM1 displayed on the display 70. The marker MA2 indicating the measurement position on the two-dimensional B-mode image IM1 is an example of first measurement position information which is information related to the measurement position set in the two-dimensional ultrasonic image.

In the present embodiment, the user manually moves the position of the marker MA2 indicating the measurement position on the two-dimensional B-mode image IM1 via the input device 50, thereby setting the marker MA2 on the two-dimensional B-mode image IM1. Furthermore, in the example illustrated in FIG. 3, a region of interest R2 for synthesizing a two-dimensional color Doppler image is set in the two-dimensional B-mode image IM1. Note that the region of interest R2 may not be set in the two-dimensional B-mode image IM1. Furthermore, in the example illustrated in FIG. 3, the Doppler waveform WA1 is displayed on the display 70, but the Doppler waveform WA1 may not be displayed before the start of the PWD mode. In addition, in a case where the marker MA1 and the marker MA2 are markers for measuring the Doppler waveform by the PWD method, the marker MA1 and the marker MA2 are also referred to as a PWD marker in the following description.

As described above, the PWD mode scan in the PWD mode and the B-mode scan in the B-mode cannot be used together. Therefore, one of the two-dimensional B-mode image IM1 and the Doppler waveform WA1 displayed on the display 70 is displayed live (in real time), and the other one is displayed in a frozen state. Specifically, in a case where the B-mode is selected, the Doppler waveform WA1 displayed immediately before the B-mode is started is displayed as a still image, that is, the Doppler waveform WA1 is displayed in a frozen state, and the two-dimensional B-mode image IM1 is displayed live. On the other hand, in a case where the PWD mode is selected, the two-dimensional B-mode image IM1 displayed immediately before the start of the PWD mode is displayed as a still image, that is, the two-dimensional B-mode image IM1 is displayed in a frozen state, and the Doppler waveform WA1 is displayed live.

The acquisition function 383 acquires the two-dimensional ultrasonic image and the first measurement position information. Specifically, for example, the acquisition function 383 acquires the two-dimensional B-mode image IM1 as the two-dimensional ultrasonic image, and acquires the marker MA2 indicating the measurement position set on the two-dimensional B-mode image IM1 as the first measurement position information.

The calculation function 384 calculates a matching degree between the two-dimensional ultrasonic image and the first measurement position information, and the target image TA1 and the second measurement position information.

The transition control function 385 controls the transition to the Doppler mode for measuring blood flow information in the subject P based on a calculation result of the calculation function 384. For example, the transition control function 385 controls the transition to the PWD mode as the Doppler mode.

The input device 50 includes a mouse, a keyboard, a button, a panel switch, a touch command screen, a wheel, a dial, a foot switch, a trackball, a joystick, and the like, receives various setting requests from the user of the ultrasonic diagnostic apparatus 1, and transfers the received various setting requests to the apparatus main body 30.

The display 70 displays a graphical user interface (GUI) for the user of the ultrasonic diagnostic apparatus 1 to input various setting requests using the input device 50, and displays the ultrasonic image, the Doppler waveform WA1, and the like generated in the apparatus main body 30. In addition, the display 70 displays various messages in order to notify the user of a processing status of the apparatus main body 30. Furthermore, the display 70 includes a speaker and can also output sound. For example, the speaker of the display 70 outputs a predetermined sound such as a beep sound in order to notify the user of the processing status of the apparatus main body 30. The display 70 corresponds to a display unit according to the present embodiment.

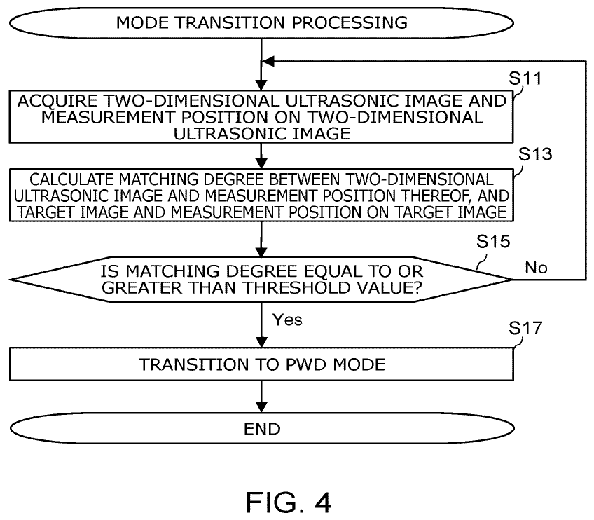

FIG. 4 is a flowchart for description of mode transition processing executed in the ultrasonic diagnostic apparatus 1 according to the first embodiment. In this mode transition processing, a two-dimensional ultrasonic image or a measurement position on the two-dimensional ultrasonic image is acquired, a matching degree between the two-dimensional ultrasonic image and the measurement position thereof, and the target image TA1 and a measurement position on the target image TA1 is calculated, it is determined whether the matching degree is equal to or greater than a threshold value, and a transition to the PWD mode is performed. For example, the mode transition processing is processing executed when the processing of setting the measurement position in the two-dimensional ultrasonic image is started in the B mode.

As illustrated in FIG. 4, first, the acquisition function 383 in the processing circuitry 38 of the apparatus main body 30 acquires the two-dimensional ultrasonic image and the measurement position on the two-dimensional ultrasonic image (step S11). Specifically, the acquisition function 383 acquires, from the image memory 36, the two-dimensional B-mode image IM1 displayed on the display 70 as the two-dimensional ultrasonic image and the marker MA2 set on the two-dimensional B-mode image IM1 as the measurement position.

Next, as illustrated in FIG. 4, the calculation function 384 in the processing circuitry 38 of the apparatus main body 30 calculates a matching degree between the two-dimensional ultrasonic image and the measurement position thereof, and the target image TA1 and the measurement position on the target image TA1 (step S13). Specifically, the calculation function 384 calculates a matching degree between the two-dimensional B-mode image IM1 acquired by the acquisition function 383 in step S11 and the marker MA2 set on the two-dimensional B-mode image IM1, and the target image TA1 stored in the storage circuitry 37 and the marker MA1 set on the target image TA1. More specifically, the calculation function 384 calculates a matching degree between the two-dimensional B-mode image IM1 and the target image TA1 and a matching degree between the position of the marker MA2 set on the two-dimensional B-mode image IM1 and the position of the marker MA1 set on the target image TA1.

Next, as illustrated in FIG. 4, the transition control function 385 in the processing circuitry 38 of the apparatus main body 30 determines whether the matching degree is equal to or greater than a threshold value (step S15). Specifically, the transition control function 385 determines whether each of the matching degree between the images and the matching degree between the measurement positions, which are each calculated in step S13, is equal to or greater than the threshold value. Then, in step S15, in a case where the matching degree is not equal to or greater than the threshold value (step S15: No), the processing returns to step S11 described above, and the processing of acquiring the two-dimensional ultrasonic image and the measurement position on the two-dimensional ultrasonic image (step S11) and the processing of calculating the matching degree (step S13) are repeated and standby is performed.

On the other hand, in step S15, in a case where the matching degree is equal to or greater than the threshold value (step S15: Yes), the transition control function 385 in the processing circuitry 38 of the apparatus main body 30 causes the transition to the PWD mode (step S17). Specifically, the transition control function 385 transitions from the B mode to the PWD mode. In addition, when the transition control function 385 makes the transition to the PWD mode, the display control function 382 controls the display 70 so that the live display of the two-dimensional B-mode image IM1 is frozen and the Doppler waveform WA1, which is an example of the blood flow information, is displayed live.

In step S17, the mode transition processing ends by transitioning to the PWD mode.

As described above, in the ultrasonic diagnostic apparatus 1, the two-dimensional ultrasonic image and the measurement position on the two-dimensional ultrasonic image are acquired, the matching degree between the two-dimensional ultrasonic image and the measurement position thereof, and the target image TA1 and the measurement position of the target image TA1 is calculated, it is determined whether the matching degree is equal to or greater than the threshold value, and in a case where the matching degree is equal to or greater than the threshold value, the transition to the PWD mode is performed. Therefore, the ultrasonic diagnostic apparatus 1 assists the user's operation for transitioning to the PWD mode, so that the user can concentrate on drawing the two-dimensional ultrasonic image.

### Second Embodiment

In the ultrasonic diagnostic apparatus 1 according to the first embodiment described above, a target image is, for example, an ultrasonic image in which a measurement position is set in the frozen state of the live display of the two-dimensional B-mode image IM1, but the target image is not limited thereto.

The target image may be, for example, an ultrasonic image in which a measurement position is set in the past ultrasonic inspection. Specifically, this target image may be an ultrasonic image in which a marker indicating the measurement position is set by the user when the Doppler waveform WA1 is measured in the past ultrasonic inspection of the subject P. This target image is different from the target image in the first embodiment in that the target image is not a target image generated during the ultrasonic inspection of the subject P but a target image generated in the past ultrasonic inspection of the subject P. That is, the storage circuitry 37 may store, as the target image, the ultrasonic image in which the marker indicating the measurement position is set by the user when the Doppler waveform WA1 is measured in the past ultrasonic inspection of the subject P, which is an ultrasonic image in which the measurement position is set. In addition, the storage circuitry 37 may store the marker indicating the measurement position set on the ultrasonic image as the second measurement position information together with the ultrasonic image in which the marker indicating the measurement position is set by the user when the Doppler waveform WA1 is measured in the past ultrasonic inspection of the subject P.

By using the target image and the second measurement position information for calculation of the matching degree, in a case where the user desires to measure the Doppler waveform WA1 at the same position as the position at which the Doppler waveform was measured in the past ultrasonic inspection in a plurality of visits, periodic medical examinations, reexaminations, and the like, it is possible to automatically transition to the PWD mode by drawing an ultrasonic image having a high matching degree with an ultrasonic image in which the marker indicating the measurement position is set by the user when the Doppler waveform WA1 is measured in the past ultrasonic inspection which is the target image TA1, and setting the marker at a position having a high matching degree with the position of the marker indicating the measurement position when the Doppler waveform WA1 is measured in the past ultrasonic inspection in the drawn ultrasonic image. Therefore, by assisting the user's operation for the ultrasonic diagnostic apparatus 1 to transition to the PWD mode, the user can concentrate on drawing the two-dimensional ultrasonic image.

Furthermore, the target image may be, for example, an ultrasonic image in which a measurement position immediately before transitioning to the PWD mode is set. Specifically, for example, the target image may be an ultrasonic image displayed on the display 70 immediately before transitioning to the PWD mode and in which a marker indicating a measurement position is set by the user. That is, the storage circuitry 37 may store, as the target image, the ultrasonic image that is displayed on the display 70 immediately before transitioning to the PWD mode and in which the marker indicating the measurement position is set by the user. In addition, the storage circuitry 37 may store, as the second measurement position information, a marker indicating the measurement position set on the ultrasonic image together with the ultrasonic image that is displayed on the display 70 immediately before transitioning to the PWD mode and in which the marker indicating the measurement position is set by the user.

By using the target image TA1 and the second measurement position information for calculation of the matching degree, in a case where the subject P moves during the measurement of the Doppler waveform in the PWD mode and processing is performed again from processing of drawing the cross section intended by the user, it is possible to automatically transition to the PWD mode by setting the marker indicating the measurement position at a position having a high matching degree with the position of the marker indicating the measurement position set by the user immediately before the transition to the PWD mode in the drawn ultrasonic image while drawing the ultrasonic image having a high matching degree with the ultrasonic image that is displayed on the display 70 and in which the marker indicating the measurement position is set by the user immediately before transitioning to the PWD mode. Therefore, by assisting the user's operation for the ultrasonic diagnostic apparatus 1 to transition to the PWD mode, the user can concentrate on drawing the two-dimensional ultrasonic image.

### Third Embodiment

In the ultrasonic diagnostic apparatus 1 according to the first embodiment described above, the position of the marker MA2 in the two-dimensional ultrasonic image is manually set by the user, but the present invention is not limited thereto. In a third embodiment, in a case where the matching degree between the images is equal to or greater than the threshold value and the matching degree between the measurement positions is equal to or less than the threshold value, the position of the marker MA2 set in the two-dimensional ultrasonic image can be moved so that the matching degree between the measurement positions becomes higher (becomes equal to or greater than the threshold value). Hereinafter, a case in which this modification is applied to the first embodiment will be referred to as a third embodiment, and portions different from those of the above-described first embodiment will be described. In the following, a case in which this modification is applied to the first embodiment will be described, but this modification is also applicable to the second embodiment described above.

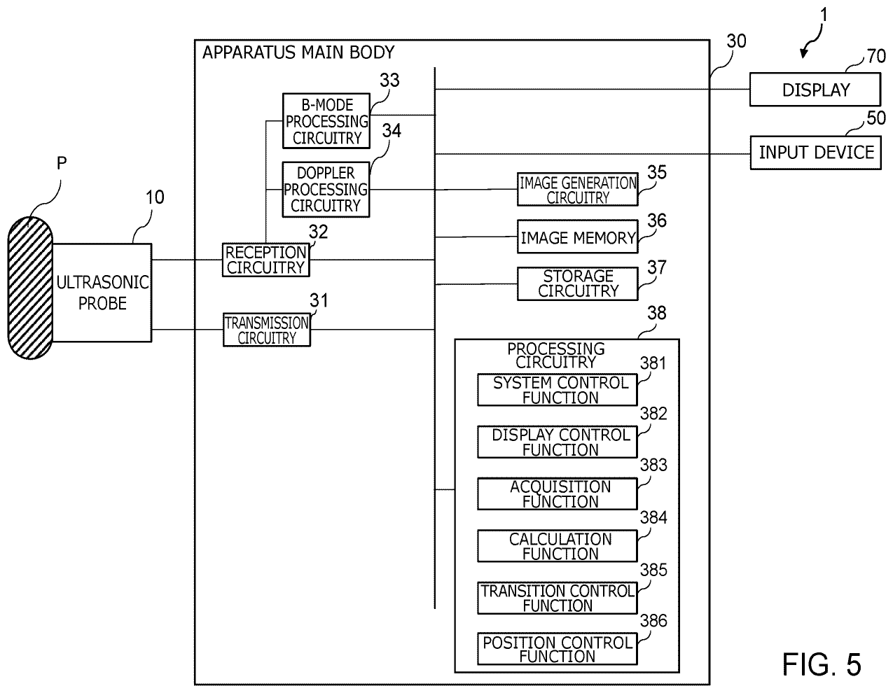

FIG. 5 is a block diagram illustrating an example of a configuration of the ultrasonic diagnostic apparatus 1 according to the third embodiment, and is a diagram corresponding to FIG. 1. As illustrated in FIG. 5, the ultrasonic diagnostic apparatus 1 according to the present embodiment is configured by adding a position control function 386 to the processing circuitry 38, with respect to the ultrasonic diagnostic apparatus 1 according to the first embodiment. Configurations and functions other than the position control function 386 are the same as those in FIG. 1 of the first embodiment described above, and thus the description thereof will be omitted.

The position control function 386 controls a position of a marker indicating a measurement position set on a two-dimensional ultrasonic image so that a matching degree between first measurement position information and second measurement position information becomes high.

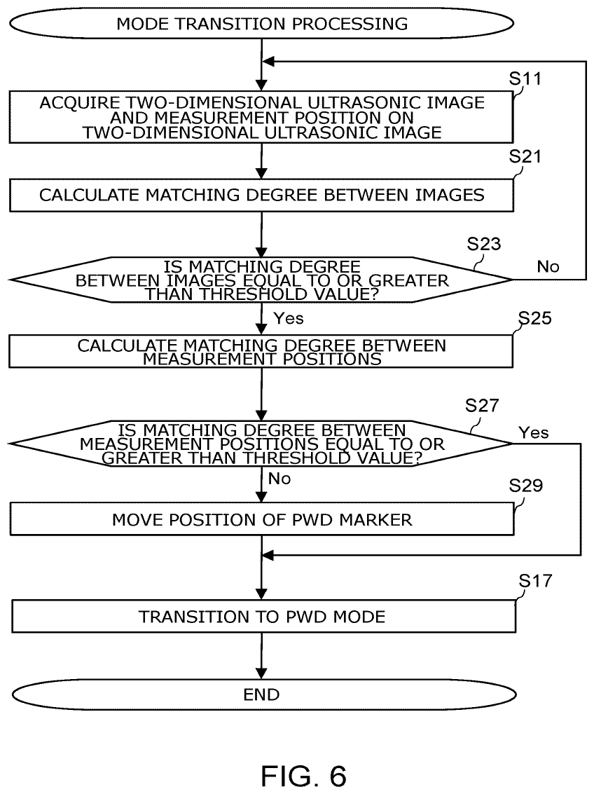

FIG. 6 is a flowchart for description of mode transition processing executed in the ultrasonic diagnostic apparatus 1 according to the third embodiment, and is a diagram corresponding to FIG. 4. In the mode transition processing according to the third embodiment, a matching degree between images is calculated, it is determined whether the matching degree between the images is equal to or greater than a threshold value, a matching degree between measurement positions is calculated, it is determined whether the matching degree between the measurement positions is equal to or greater than a threshold value, the position of a PWD marker is controlled, and the mode transitions to a PWD mode. For example, mode transition processing is processing executed in a case where processing of setting the measurement position in the two-dimensional ultrasonic image is started. Note that processing in step S11 illustrated in FIG. 6 is the same as that in FIG. 4, and thus the description thereof will be omitted.

Next, as illustrated in FIG. 6, the calculation function 384 in the processing circuitry 38 of the apparatus main body 30 calculates a matching degree between images (step S21). Specifically, the calculation function 384 calculates a matching degree between the two-dimensional B-mode image IM1 acquired by the acquisition function 383 in step S11 and the target image TA1 stored in the storage circuitry 37.

Next, as illustrated in FIG. 6, the transition control function 385 in the processing circuitry 38 of the apparatus main body 30 determines whether the matching degree between the images is equal to or greater than the threshold value (step S23). Then, in step S23, in a case where the matching degree between the images is not equal to or greater than the threshold value (step S23: No), the processing returns to step S11 described above, and processing of acquiring a two-dimensional ultrasonic image and a measurement position on the two-dimensional ultrasonic image (step S11) and processing of calculating the matching degree between the images (step S23) are repeated and standby is performed.

On the other hand, in a case where the matching degree between the images is equal to or greater than the threshold value in step S23 (step S23: Yes), the calculation function 384 in the processing circuitry 38 of the apparatus main body 30 calculates the matching degree between the measurement positions (step S25). Specifically, the calculation function 384 calculates the matching degree between the measurement positions between the position of the marker MA2 set on the two-dimensional B-mode image IM1 and the position of the marker MA1 set on the target image TA1.

Next, as illustrated in FIG. 6, the transition control function 385 in the processing circuitry 38 of the apparatus main body 30 determines whether the matching degree between the measurement positions is equal to or greater than the threshold value (step S27). Then, in step S27, in a case where the matching degree between the measurement positions is not equal to or greater than the threshold value (step S27: No), the position control function 386 in the processing circuitry 38 of the apparatus main body 30 moves the position of the PWD marker (step S29).

Specifically, the position control function 386 moves the position of the marker MA2 set on the two-dimensional B-mode image IM1 so that a matching degree between the first measurement position information and the second measurement position information becomes high. More specifically, for example, the position control function 386 moves the position of the marker MA2 set on the two-dimensional B-mode image IM1 so as to be the same as the position of the marker MA1 set on the target image TA1.

After step S29 or in a case where the matching degree between the measurement positions is equal to or greater than the threshold value in step S27 (step S27: No), the processing in step S17 is the same as that of the first embodiment described above, and thus the description thereof will be omitted. Then, in step S17, the mode transition processing is terminated by transitioning to the PWD mode.

As described above, in the ultrasonic diagnostic apparatus 1, the two-dimensional ultrasonic image and the measurement position on the two-dimensional ultrasonic image are acquired, the matching degree between the images is calculated, it is determined whether the matching degree between the images is equal to or greater than the threshold value, the matching degree between the measurement positions is calculated in a case where the matching degree between the images is equal to or greater than the threshold value, it is determined whether the matching degree between the measurement positions is equal to or greater than the threshold value, the position of the PWD marker is controlled in a case where the matching degree between the measurement positions is equal to or less than the threshold value, and the transition to the PWD mode is performed. Therefore, the ultrasonic diagnostic apparatus 1 assists the user's operation for setting the PWD marker in the two-dimensional ultrasonic image and the user's operation for transitioning to the PWD mode, so that the user can concentrate on drawing the two-dimensional ultrasonic image.

### Fourth Embodiment

In the ultrasonic diagnostic apparatus 1 according to the first embodiment described above, it is also possible to notify the user of the calculated matching degree. Hereinafter, a case in which this modification is applied to the first embodiment will be referred to as a fourth embodiment, and portions different from those of the above-described first embodiment will be described. In the following, a case in which this modification is applied to the first embodiment will be described, but this modification is also applicable to the second and third embodiments described above.

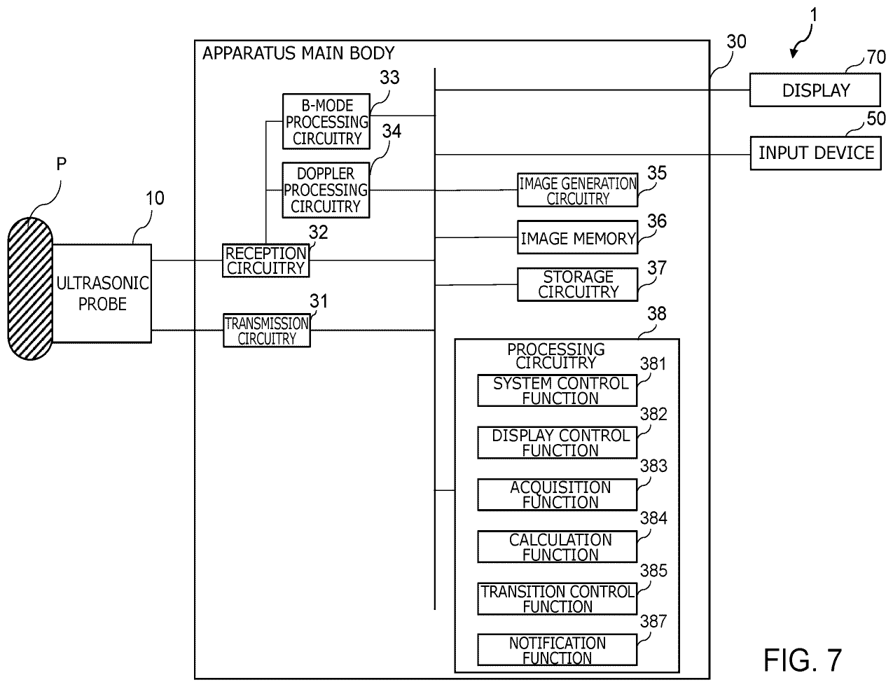

FIG. 7 is a block diagram illustrating an example of a configuration of the ultrasonic diagnostic apparatus 1 according to the fourth embodiment, and is a diagram corresponding to FIG. 1. As illustrated in FIG. 7, the ultrasonic diagnostic apparatus 1 according to the present embodiment is configured by adding a notification function 387 to the processing circuitry 38, with respect to the ultrasonic diagnostic apparatus 1 according to the first embodiment. Note that configurations and functions other than the notification function 387 are the same as those in FIG. 1 of the first embodiment described above, and thus the description thereof will be omitted.

The notification function 387 notifies the matching degree between the two-dimensional ultrasonic image and the measurement position thereof, and the target image TA1 and the measurement position on the target image TA1. Specifically, the notification function 387 notifies the matching degree between the two-dimensional ultrasonic image and the measurement position thereof, and the target image TA1 and the measurement position on the target image TA1 by one of color, number, and sound, or a combination of two or more of color, number, and sound.

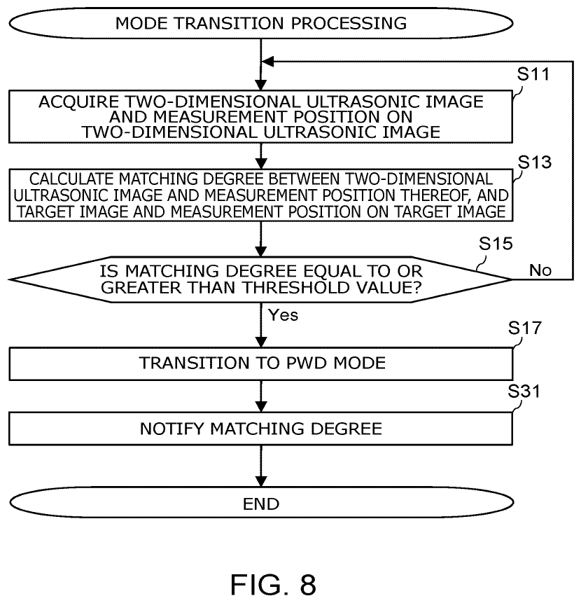

FIG. 8 is a flowchart for description of mode transition processing executed in the ultrasonic diagnostic apparatus 1 according to the fourth embodiment, and is a diagram corresponding to FIG. 4. In the mode transition processing according to the fourth embodiment, the two-dimensional ultrasonic image and the measurement position thereof are acquired, the matching degree between the two-dimensional ultrasonic image and the measurement position thereof, and the target image TA1 and the measurement position on the target image TA1 is calculated, it is determined whether the matching degree is equal to or greater than the threshold value, a transition to the PWD mode is performed, and a notification of the calculated matching degree is performed. For example, mode transition processing is processing executed in a case where processing of setting the measurement position in the two-dimensional ultrasonic image is started. Note that the processing from step S11 to step S17 illustrated in FIG. 8 is the same as that in FIG. 4, and thus the description thereof will be omitted.

Next, as illustrated in FIG. 8, the notification function 387 in the processing circuitry 38 of the apparatus main body 30 notifies the matching degree (step S31). Specifically, the notification function 387 notifies the user of the matching degree between the two-dimensional ultrasonic image and the measurement position thereof, and the target image TA1 and the measurement position on the target image TA1, which is calculated by the calculation function 384 in step S13.

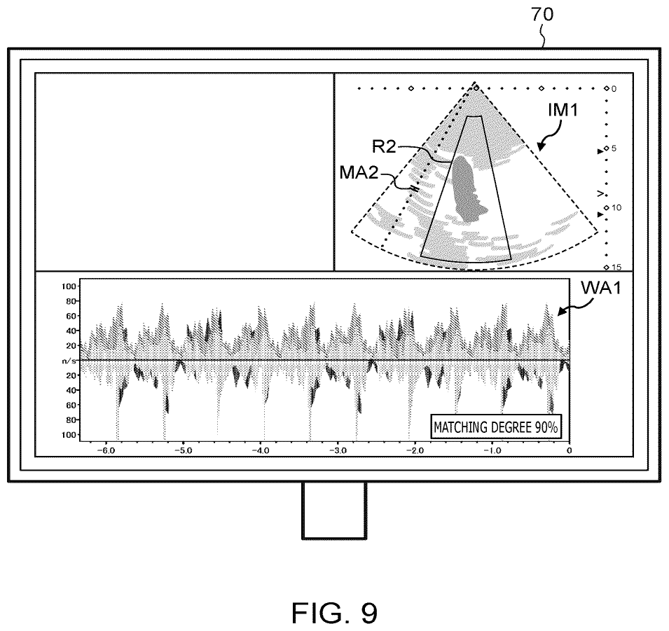

FIG. 9 is a diagram illustrating an example of the two-dimensional ultrasonic image and the Doppler waveform displayed on the display 70 in the ultrasonic diagnostic apparatus 1 according to the fourth embodiment, and is a diagram corresponding to FIG. 3. In the example illustrated in FIG. 9, the display control function 382 causes the display 70 to display the two-dimensional B-mode image IM1 as the two-dimensional ultrasonic image, the Doppler waveform WA1, and the marker MA2 indicating the measurement position on the two-dimensional B-mode image IM1, and the notification function 387 notifies the user by displaying “90%”, which is a number of a matching degree between the two-dimensional B-mode image IM1 and the measurement position thereof, and the target image TA1 and the measurement position on the target image TA1, in the vicinity of the Doppler waveform WA1.

In step S31, the mode transition processing is terminated by notifying the matching degree.

As described above, in the ultrasonic diagnostic apparatus 1, the two-dimensional ultrasonic image and the measurement position on the two-dimensional ultrasonic image are acquired, the matching degree between the two-dimensional ultrasonic image and the measurement position thereof, and the target image TA1 and the measurement position on the target image TA1 is calculated, it is determined whether the matching degree is equal to or greater than the threshold value, and in a case where the matching degree is equal to or greater than the threshold value, the mode is caused to transition to the PWD mode, and the calculated matching degree is notified to the user. Therefore, the user can consider whether the PWD marker can be set at the intended measurement position on the intended two-dimensional ultrasonic image.

Note that, in the above-described fourth embodiment, the notification function 387 notifies the user of one matching degree between the two-dimensional ultrasonic image and the measurement position thereof, and the target image TA1 and the measurement position on the target image TA1. However, in a case where each of the matching degree between the two-dimensional ultrasonic image and the target image TA1 and the matching degree between the measurement position on the two-dimensional ultrasonic image and the measurement position on the target image TA1 is calculated, each of the matching degree between the images and the matching degree between the measurement positions may be notified to the user.

Furthermore, in the above-described fourth embodiment, the color and the number regarding the matching degree notified by the notification function 387 may be stored together in the image memory 36 or the storage circuitry 37 when the two-dimensional ultrasonic image is stored. By storing the color and number related to the matching degree with the two-dimensional ultrasonic image in this manner, it is possible to use the color and number as a guide for examining whether the PWD marker has been arranged at the intended measurement position when the user looks back on the inspection result later.

Furthermore, in the above-described fourth embodiment, in a case where the notification function 387 notifies a color regarding the matching degree, for example, the notification function 387 may notify the user of the color regarding the matching degree by changing a color of a frame line of the two-dimensional B-mode image or the Doppler waveform according to the matching degree.

### Fifth Embodiment

In the ultrasonic diagnostic apparatus 1 according to each embodiment described above, the calculation function can calculate the matching degree between the two-dimensional ultrasonic image and the first measurement position information, and the target image TA1 and the second measurement position information, and can also calculate the matching degree between the position of the ultrasonic probe 10 when the target image TA1 is generated and the current position of the ultrasonic probe 10. Hereinafter, a case in which this modification is applied to the first embodiment will be referred to as a fifth embodiment, and portions different from those of the above-described first embodiment will be described. In the following, a case in which this modification is applied to the first embodiment will be described, but this modification is also applicable to the second to fourth embodiments described above.

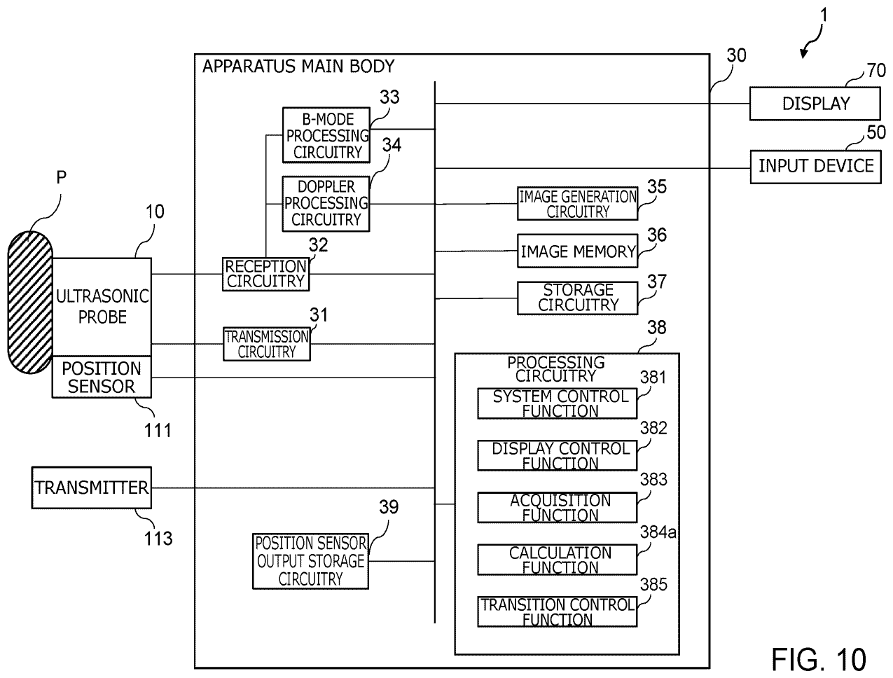

FIG. 10 is a block diagram illustrating an example of a configuration of the ultrasonic diagnostic apparatus 1 according to the fifth embodiment, and is a diagram corresponding to FIG. 1. As illustrated in FIG. 10, in the ultrasonic diagnostic apparatus 1 according to the present embodiment, the calculation function of the processing circuitry 38 is different from that of the first embodiment described above, and thus is denoted as a calculation function 384a. The ultrasonic diagnostic apparatus according to the present embodiment is configured by adding a position sensor 111, a transmitter 113, and a position sensor output storage circuitry 39 to the ultrasonic diagnostic apparatus 1 according to the first embodiment. Note that configurations and functions other than the calculation function 384a, the position sensor 111, the transmitter 113, and the position sensor output storage circuitry 39 are the same as those in FIG. 1 of the first embodiment described above, and thus the description thereof will be omitted.

The position sensor 111 and the transmitter 113 are devices (position detection systems) for acquiring position information of the ultrasonic probe 10. The position sensor 111 detects the position of the ultrasonic probe 10. Specifically, for example, the position sensor 111 is a magnetic sensor attached to the ultrasonic probe 10. Furthermore, for example, the transmitter 113 is a device that is disposed at an arbitrary position and forms a magnetic field toward the outside around the own device.

The position sensor 111 detects a three-dimensional magnetic field formed by the transmitter 113. Then, the position sensor 111 calculates a position (coordinates) and a direction (angle) of the own device in a space with the transmitter 113 as an origin based on information of the detected magnetic field, and outputs the calculated position and direction to the position sensor output storage circuitry 39.

In the present embodiment, a case in which the position information of the ultrasonic probe 10 is acquired by the above-described position detection system has been described. The configuration of the position detection system is not limited thereto. That is, the configuration of the position detection system is arbitrary, and may be configured by, for example, a gyro sensor, an acceleration sensor, or the like.

The position sensor output storage circuitry 39 stores the position of the ultrasonic probe 10, which is output from the position sensor 111. For example, the position sensor output storage circuitry 39 stores the position of the ultrasonic probe 10, which is output from the position sensor 111, in time series, or stores the position of the ultrasonic probe 10 when the target image TA1 detected by the position sensor 111 is generated. The position of the ultrasonic probe 10 when the target image TA1 is generated is, for example, the position of the ultrasonic probe 10 when the live display of the two-dimensional B-mode image displayed live on the display 70 is frozen.

The calculation function 384a calculates the matching degree between the two-dimensional ultrasonic image and the first measurement position information, and the target image TA1 and the second measurement position information, and the matching degree between the position of the ultrasonic probe 10 when the target image TA1 detected by the position sensor 111 is generated and the current position of the ultrasonic probe 10 detected by the position sensor 111.

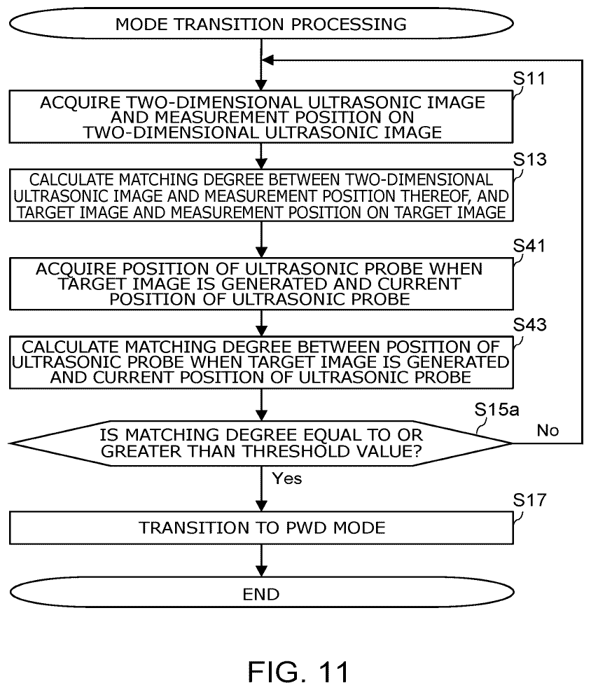

FIG. 11 is a flowchart for description of mode transition processing executed in the ultrasonic diagnostic apparatus 1 according to the fifth embodiment, and is a diagram corresponding to FIG. 4. In the mode transition processing according to the fifth embodiment, the two-dimensional ultrasonic image and the measurement position are acquired, the matching degree between the two-dimensional ultrasonic image and the measurement position thereof, and the target image TA1 and the measurement position on the target image TA1 is calculated, the position of the ultrasonic probe 10 when the target image TA1 is generated and the current position of the ultrasonic probe 10 are acquired, the matching degree between the position of the ultrasonic probe 10 when the target image TA1 is generated and the current position of the ultrasonic probe 10 is calculated, it is determined whether the matching degree is equal to or greater than the threshold value, and the mode transitions to the PWD mode. For example, mode transition processing is processing executed in a case where processing of setting the measurement position in the two-dimensional ultrasonic image is started. Note that the processing in steps S11 and S13 illustrated in FIG. 11 is the same as that in FIG. 4, and thus the description thereof will be omitted.

Next, as illustrated in FIG. 11, the acquisition function 383 in the processing circuitry 38 of the apparatus main body 30 acquires the position of the ultrasonic probe 10 when the target image TA1 is generated and the current position of the ultrasonic probe 10 (step S41). Specifically, the acquisition function 383 acquires the position of the ultrasonic probe 10 when the target image TA1 is generated and the current position of the ultrasonic probe 10 from the position sensor output storage circuitry 39.

Next, as illustrated in FIG. 11, the calculation function 384a in the processing circuitry 38 of the apparatus main body 30 calculates the matching degree between the position of the ultrasonic probe 10 when the target image TA1 is generated and the current position of the ultrasonic probe 10 (step S43).

Next, as illustrated in FIG. 11, the transition control function 385a in the processing circuitry 38 of the apparatus main body 30 determines whether the matching degree is equal to or greater than the threshold value (step S15a). Specifically, the transition control function 385a determines whether each of the matching degree between the two-dimensional B-mode image IM1 and the measurement position thereof and the target image TA1 and the measurement position on the target image TA1, which is calculated in step S13, and the matching degree between the position of the ultrasonic probe 10 when the target image TA1 is generated and the current position of the ultrasonic probe 10, which is calculated in step S43, is equal to or greater than the threshold value. More specifically, the transition control function 385a determines whether each of the matching degree between the two-dimensional B-mode image IM1 and the measurement position thereof and the target image TA1 and the measurement position on the target image TA1, which is calculated in step S13, and the matching degree between the position of the ultrasonic probe 10 when the target image TA1 is generated and the current position of the ultrasonic probe 10, which is calculated in step S43, is equal to or greater than the threshold value.

Then, in step S15a, in a case where the matching degree is not equal to or greater than the threshold value (step S15a: No), the processing returns to step S11 described above, and the processing of acquiring the two-dimensional ultrasonic image and the measurement position on the two-dimensional ultrasonic image (step S11), the processing of calculating the matching degree between the two-dimensional ultrasonic image and the measurement position thereof, and the target image TA1 and the measurement position on the target image TA1 (step S13), the processing of acquiring the position of the ultrasonic probe 10 when the target image TA1 is generated and the current position of the ultrasonic probe 10 (step S41), and the processing of calculating the matching degree between the position of the ultrasonic probe 10 when the target image TA1 is generated and the current position of the ultrasonic probe 10 (step S43) are repeated and standby is performed. Note that the processing in step S17 after the matching degree is equal to or greater than the threshold value in step S15a (step S15a: Yes) is the same as that in the first embodiment described above, and thus the description thereof will be omitted. Then, in step S17, the mode transition processing is terminated by transitioning to the PWD mode.

As described above, in the ultrasonic diagnostic apparatus 1, the two-dimensional ultrasonic image and the measurement position on the two-dimensional ultrasonic image are acquired, the matching degree between the two-dimensional ultrasonic image and the measurement position thereof, and the target image TA1 and the measurement position on the target image TA1 is calculated, the position of the ultrasonic probe 10 when the target image TA1 is generated and the current position of the ultrasonic probe 10 are acquired, the matching degree between the position of the ultrasonic probe 10 when the target image TA1 is generated and the current position of the ultrasonic probe 10 is calculated, it is determined whether the matching degree is equal to or greater than the threshold value, and in a case where the matching degree is equal to or greater than the threshold value, the mode transitions to the PWD mode. Therefore, since the ultrasonic diagnostic apparatus 1 assists the user's operation for transitioning to the PWD mode, the user can focus on drawing the two-dimensional ultrasonic image.

In addition, the ultrasonic diagnostic apparatus 1 acquires the position of the ultrasonic probe 10 when the target image TA1 is generated and the current position of the ultrasonic probe 10, calculates the matching degree between the position of the ultrasonic probe 10 when the target image TA1 is generated and the current position of the ultrasonic probe 10, and also determines whether the matching degree between the position of the ultrasonic probe 10 when the target image TA1 is generated and the current position of the ultrasonic probe 10 is equal to or greater than the threshold value. Therefore, the mode can transition to the PWD mode at a more accurate position of the ultrasonic probe 10.

In the ultrasonic diagnostic apparatus 1 according to the fifth embodiment described above, the position sensor output storage circuitry 39 is provided, and the position of the ultrasonic probe 10, which is output from the position sensor 111, is stored in the position sensor output storage circuitry 39, but the position sensor output storage circuitry 39 may not be provided. In this case, the position of the ultrasonic probe 10, which is output from the position sensor 111, may be stored in the storage circuitry 37.

Further, in the ultrasonic diagnostic apparatus 1 according to the fifth embodiment described above, it is determined whether each of the matching degree between the two-dimensional ultrasonic image and the measurement position thereof and the target image TA1 and the measurement position on the target image TA1, which is calculated in step S13, and the matching degree between the position of the ultrasonic probe 10 when the target image TA1 is generated and the current position of the ultrasonic probe 10, which is calculated in step S43, is equal to or greater than one threshold value. However, in the ultrasonic diagnostic apparatus 1 according to the fifth embodiment described above, different threshold values may be set for each of the matching degree between the target image TA1 and the measurement position on the target image TA1 and the matching degree between the position of the ultrasonic probe 10 when the target image TA1 is generated and the current position of the ultrasonic probe 10, which is calculated in step S43, and It may be determined whether each of the matching degree between the two-dimensional ultrasonic image and the measurement position thereof and the target image TA1 and the measurement position on the target image TA1, which is calculated in step S13, and the matching degree between the position of the ultrasonic probe 10 when the target image TA1 is generated and the current position of the ultrasonic probe 10, which is calculated in step S43, is equal to or greater than each threshold value.

Furthermore, in the fifth embodiment described above, the position of the ultrasonic probe 10 when the target image TA1 is generated is not limited to the position of the ultrasonic probe 10 when the live display of the two-dimensional B-mode image live-displayed on the display 70 is frozen, and in a case where the target image is an ultrasonic image in which the measurement position is set in the past ultrasonic inspection, the position of the ultrasonic probe 10 when the target image TA1 is generated may be the position of the ultrasonic probe 10 when the measurement position is set in the ultrasonic probe in the past ultrasonic inspection, and in a case where the target image is an ultrasonic image in which the measurement position immediately before transitioning to the PWD mode is set, the position of the ultrasonic probe 10 when the target image TA1 is generated may be the position of the ultrasonic probe 10 immediately before transitioning to the PWD mode.

### Sixth Embodiment

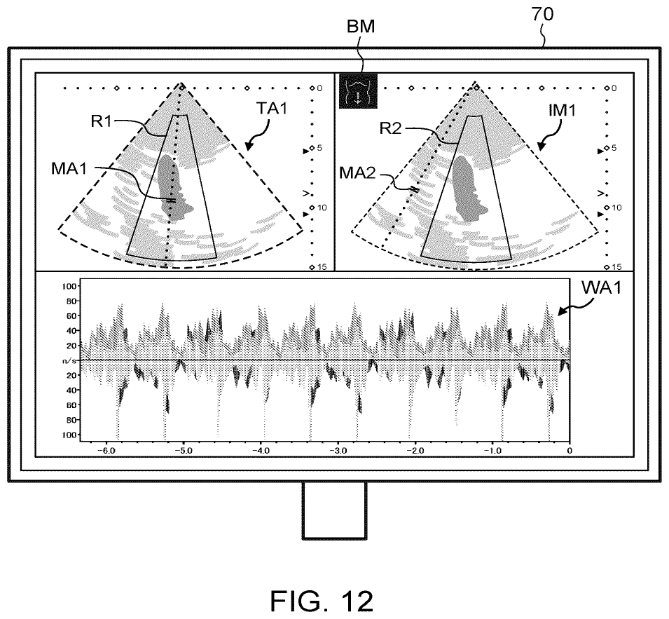

In the ultrasonic diagnostic apparatus 1 according to each of the above-described embodiments, the display control function 382 can display at least one of the target image TA1 and a body mark related to the two-dimensional ultrasonic image on the display 70 together with the two-dimensional ultrasonic image and the Doppler waveform WA1 serving as blood flow information. FIG. 12 is a diagram illustrating an example of the two-dimensional ultrasonic image displayed on the display 70, the Doppler waveform WA1, and the target image TA1 in the ultrasonic diagnostic apparatus 1 according to the sixth embodiment, and is a diagram corresponding to FIG. 3.

As illustrated in FIG. 12, the display control function 382 controls the display 70 to display the target image TA1 and a body mark BM related to the two-dimensional ultrasonic image together with the two-dimensional B-mode image IM1 and the Doppler waveform WA1.

Specifically, in the example illustrated in FIG. 12, the display control function 382 displays the target image TA1 and the body mark BM related to the two-dimensional ultrasonic image on the display 70 side by side with the two-dimensional B-mode image IM1 and the Doppler waveform WA1. More specifically, in the example illustrated in FIG. 12, the display control function 382 displays the target image TA1 on the upper side of the Doppler waveform WA1 and on the left side of the two-dimensional B-mode image IM1, and displays the body mark related to the two-dimensional ultrasonic image on the upper side of the two-dimensional B-mode image IM1.

Note that, in the example illustrated in FIG. 12, the display control function 382 causes the display 70 to display the target image TA1 and the body mark BM related to the two-dimensional ultrasonic image side by side with the two-dimensional B-mode image IM1 and the Doppler waveform WA1. However, the display control function 382 may cause the display 70 to display the target image TA1 and the body mark BM in a state of being superimposed on at least one of the two-dimensional B-mode image IM1 and the Doppler waveform WA1.

In the example illustrated in FIG. 12, the display control function 382 displays both the target image TA1 and the body mark BM on the display 70. However, the display control function 382 may display either the target image TA1 or the body mark BM on the display 70. That is, the display control function 382 may control the display 70 to display at least one of the target image TA1 and the body mark BM together with the two-dimensional B-mode image IM1 and the Doppler waveform WA1.

In the example illustrated in FIG. 12, the display control function 382 displays the target image TA1 on the upper side of the Doppler waveform WA1 and on the left side of the two-dimensional B-mode image IM1, and displays the body mark BM on the upper side of the two-dimensional B-mode image. However, the display positions of the target image TA1 and the body mark BM on the display 70 are not limited thereto. That is, the display positions of the target image TA1 and the body mark BM are arbitrary.

Furthermore, in the example illustrated in FIG. 12, the display control function 382 displays the target image TA1 and the body mark BM on the display 70 together with the two-dimensional B-mode image IM1 and the Doppler waveform WA1. However, the content displayed on the display 70 together with the two-dimensional B-mode image IM1 and the Doppler waveform WA1 by the display control function 382 is not limited thereto. That is, the content to be displayed on the display 70 is arbitrary, and an output result of the position sensor 111 may be displayed on the display 70.

### Seventh Embodiment

In the ultrasonic diagnostic apparatus 1 according to each embodiment described above, the transition control function 385 controls the transition from the mode for generating the two-dimensional ultrasonic image to the Doppler mode. However, the transition control function can control the transition from the mode for generating the two-dimensional ultrasonic image to the Doppler mode, and can also control the transition from the Doppler mode to the mode for generating the two-dimensional ultrasonic image. Hereinafter, a case in which this modification is applied to the first embodiment will be referred to as a seventh embodiment, and portions different from those of the above-described first embodiment will be described. In the following, a case in which this modification is applied to the first embodiment will be described, but this modification is also applicable to the second to sixth embodiments described above.

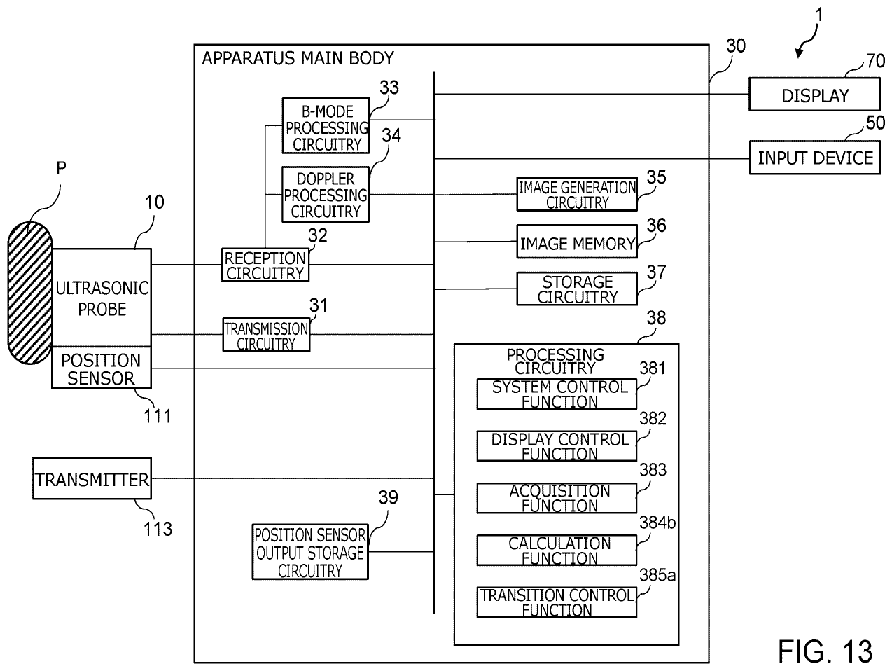

FIG. 13 is a block diagram illustrating an example of a configuration of the ultrasonic diagnostic apparatus 1 according to the seventh embodiment, and is a diagram corresponding to FIG. 1. As illustrated in FIG. 13, in the apparatus main body 30 of the ultrasonic diagnostic apparatus 1 according to the present embodiment, the calculation function and the transition control function of the processing circuitry 38 are different from those of the first embodiment described above, and thus are denoted as a calculation function 384b and a transition control function 385a. In addition, the ultrasonic diagnostic apparatus 1 according to the present embodiment is configured by adding the position sensor 111, the transmitter 113, and the position sensor output storage circuitry 39 to the ultrasonic diagnostic apparatus 1 according to the first embodiment described above. Note that the configurations of the position sensor 111, the transmitter 113, and the position sensor output storage circuitry 39 are the same as the configurations of the position sensor 111, the transmitter 113, and the position sensor output storage circuitry 39 according to the fifth embodiment described above, and thus, the description thereof will be omitted. In addition, since configurations and functions other than the calculation function 384b, the transition control function 385a, the position sensor 111, the transmitter 113, and the position sensor output storage circuitry 39 are the same as those in FIG. 1 of the first embodiment described above, the description thereof will be omitted.

The calculation function 384b according to the present embodiment calculates a matching degree between the two-dimensional ultrasonic image and the first measurement position information, and the target image TA1 in which the measurement position is set and the second measurement position information related to the measurement position set in the target image TA1, and calculates a difference between the position of the ultrasonic probe 10 at the time of transitioning to the Doppler mode detected by the position sensor 111 and the current position of the ultrasonic probe, which is detected by the position sensor 111.

In addition, the transition control function 385a according to the present embodiment determines whether the difference between the position of the ultrasonic probe 10 at the time of transitioning to the Doppler mode, which is detected by the position sensor 111, and the current position of the ultrasonic probe, which is detected by the position sensor 111, exceeds a predetermined threshold value, and controls transition to the Doppler mode for measuring blood flow information in the subject P and also controls transition from the Doppler mode to a mode for generating the two-dimensional ultrasonic image in a case where the difference exceeds the predetermined threshold value. The mode for generating the two-dimensional ultrasonic image is, for example, a B mode, a color Doppler mode, an M mode, or the like. Hereinafter, the present embodiment will be described by exemplifying a case in which the mode for generating the two-dimensional ultrasonic image is the B-mode.

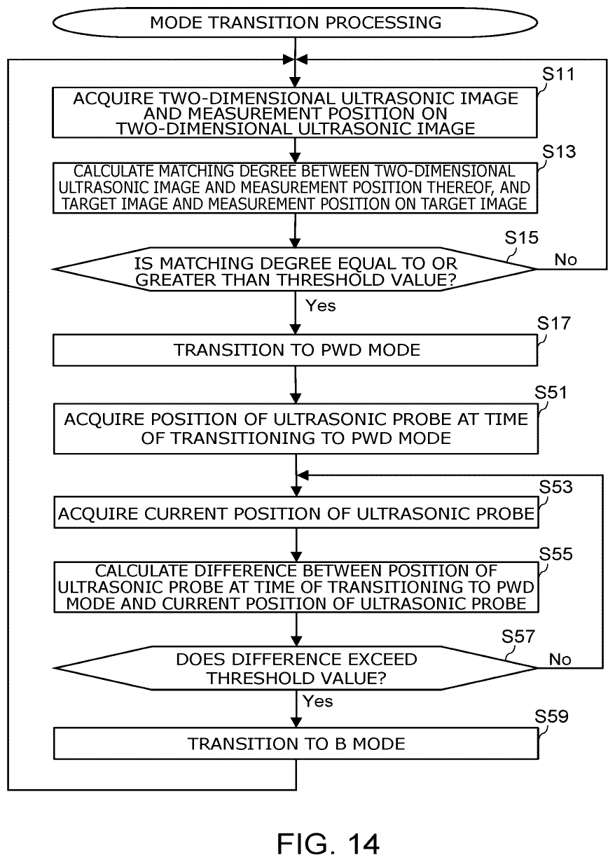

FIG. 14 is a flowchart for description of mode transition processing executed in the ultrasonic diagnostic apparatus 1 according to the seventh embodiment, and is a diagram corresponding to FIG. 4. In the mode transition processing according to the seventh embodiment, the two-dimensional ultrasonic image and the measurement position are acquired, the matching degree between the two-dimensional ultrasonic image and the measurement position thereof, and the target image TA1 and the measurement position on the target image TA1 is calculated, it is determined whether the matching degree is equal to or greater than the threshold value, the mode transitions to the PWD mode, the position of the ultrasonic probe 10 at the time of transitioning to the PWD mode is acquired, the current position of the ultrasonic probe 10 is acquired, the difference between the position of the ultrasonic probe 10 at the time of transitioning to the PWD mode and the current position of the ultrasonic probe 10 is calculated, it is determined whether the difference exceeds the threshold value, and the mode transitions to the B mode. For example, mode transition processing is processing executed in a case where processing of setting the measurement position in the two-dimensional ultrasonic image is started. Note that the processing from step S11 to step S17 is the same as that in FIG. 4, and thus the description thereof will be omitted.

Next, as illustrated in FIG. 14, the acquisition function 383 in the processing circuitry 38 of the apparatus main body 30 acquires the position of the ultrasonic probe 10 at the time of transitioning to the PWD mode (step S51). Specifically, the acquisition function 383 acquires the position of the ultrasonic probe 10 at the time of transitioning to the PWD mode in step S17 from the position sensor output storage circuitry 39.

Next, as illustrated in FIG. 14, the acquisition function 383 in the processing circuitry 38 of the apparatus main body 30 acquires the current position of the ultrasonic probe 10 (step S53). Specifically, the acquisition function 383 acquires the current position of the ultrasonic probe 10 from the position sensor output storage circuitry 39.

Next, as illustrated in FIG. 14, the calculation function 384b in the processing circuitry 38 of the apparatus main body 30 calculates a difference between the position of the ultrasonic probe 10 at the time of transitioning to the PWD mode and the current position of the ultrasonic probe 10 (step S55). Specifically, the calculation function 384b calculates the difference between the position of the ultrasonic probe 10 at the time of transitioning to the PWD mode and the current position of the ultrasonic probe 10 based on the position of the ultrasonic probe 10 at the time of transitioning to the PWD mode, which is acquired by the acquisition function 383 in step S51, and the current position of the ultrasonic probe 10, which is acquired by the acquisition function 383 in step S53.

Next, as illustrated in FIG. 14, the transition control function 385a in the processing circuitry 38 of the apparatus main body 30 determines whether the difference exceeds a threshold value (step S57). Specifically, the transition control function 385a determines whether the difference between the position of the ultrasonic probe 10 at the time of transitioning to the PWD mode, which is calculated in step S55, and the current position of the ultrasonic probe 10 exceeds the threshold value. Then, in step S57, in a case where the difference does not exceed the threshold value (step S57: No), the processing returns to step S53 described above, and the processing of acquiring the current position of the ultrasonic probe 10 (step S53) and the processing of calculating the difference between the position of the ultrasonic probe 10 at the time of transitioning to the PWD mode and the current position of the ultrasonic probe 10 (step S45) are repeated and standby is performed.

On the other hand, in a case where the difference exceeds the threshold value in step S57 (step S57: Yes), the transition control function 385a in the processing circuitry 38 of the apparatus main body 30 transitions to the B mode (step S59). Specifically, the transition control function 385a causes a transition from the PWD mode to the B mode. Then, when the transition control function 385a causes the mode to transition to the PWD mode, the display control function 382 controls the display 70 to freeze the live display of the Doppler waveform WA1 and to cause the two-dimensional ultrasonic image to be displayed live.

The mode transition processing illustrated in FIG. 14 is repeatedly executed while the user performs the inspection, and ends when the user ends the inspection.

As described above, in the ultrasonic diagnostic apparatus 1, the two-dimensional ultrasonic image and the measurement position on the two-dimensional ultrasonic image are acquired, the matching degree between the two-dimensional ultrasonic image and the measurement position thereof and the target image TA1 and the measurement position on the target image TA1 is calculated, it is determined whether the matching degree is equal to or greater than the threshold value, the mode transitions to the PWD mode in a case where the matching degree is equal to or greater than the threshold value, the position of the ultrasonic probe 10 at the time of transitioning to the PWD mode and the current position of the ultrasonic probe 10 are acquired, the difference between the position of the ultrasonic probe 10 at the time of transitioning to the PWD mode and the current position of the ultrasonic probe 10 is calculated, it is determined whether the difference exceeds the threshold value, and the mode transitions to the B mode in a case where the difference exceeds the threshold value. Therefore, even in a case where the measurement position of the ultrasonic probe 10 deviates, it is possible to automatically start from the setting of the marker indicating the measurement position in the B mode.

Note that, in the seventh embodiment described above, the transition control function 385a makes the transition to the mode for generating the two-dimensional ultrasonic image in a case where the difference between the position of the ultrasonic probe 10 at the time of transition to the PWD mode and the current position of the ultrasonic probe 10 exceeds the threshold. However, the transition control function 385a may control the transition from the PWD mode to the mode for generating the two-dimensional ultrasonic image in a case where the blood flow signal cannot be detected in the Doppler mode. In such a case, even in a case where the blood flow signal cannot be detected due to the deviation of the position of the ultrasonic probe 10, it is possible to start from the setting of the measurement position in the mode for automatically generating the two-dimensional ultrasonic image.

### Eighth Embodiment

In the ultrasonic diagnostic apparatus 1 according to each embodiment described above, the calculation function 384 calculates the matching degree between the entire two-dimensional ultrasonic image and the first measurement position information, and the entire target image and the second measurement position information. However, it is also possible to calculate the matching degree between the image around the measurement position in the two-dimensional ultrasonic image and the first measurement position information, and the image around the measurement position in the target image TA1 and the second measurement position information. Hereinafter, a case in which this modification is applied to the first embodiment will be referred to as an eighth embodiment, and portions different from those of the above-described first embodiment will be described. In the following, a case in which this modification is applied to the first embodiment will be described, but this modification is also applicable to the second to seventh embodiments described above.

Although the configuration of the ultrasonic diagnostic apparatus 1 according to the eighth embodiment is not illustrated, since the function of the calculation function of the processing circuitry is different from that of the first embodiment, the calculation function is hereinafter referred to as a calculation function 384c. In addition, since the configuration and function other than the calculation function 384c are the same as those in FIG. 1, the description thereof will be omitted.

The calculation function 384c calculates, as the matching degree between the two-dimensional ultrasonic image and the first measurement position information, and the target image TA1 and the second measurement position information, the matching degree between the image around the first measurement position information in the two-dimensional ultrasonic image in which the measurement position is set by setting the first measurement position information and the image around the second measurement position information in the target image TA1 in which the measurement position is set by setting the second measurement position information. Specifically, the calculation function 384c calculates a matching degree between a first image in a predetermined range centered on the first measurement position information in the two-dimensional ultrasonic image in which the measurement position is set by setting the first measurement position information and a second image in a predetermined range centered on the second measurement position information in the target image TA1 in which the measurement position is set by setting the second measurement position information.

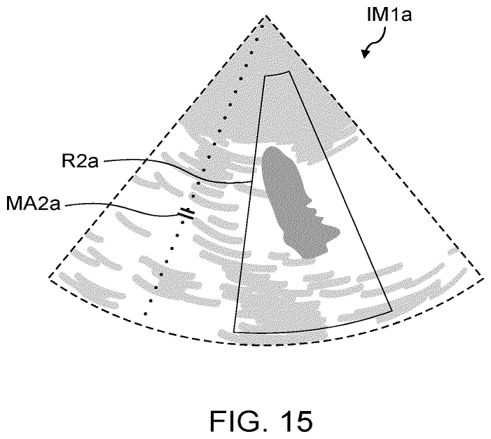

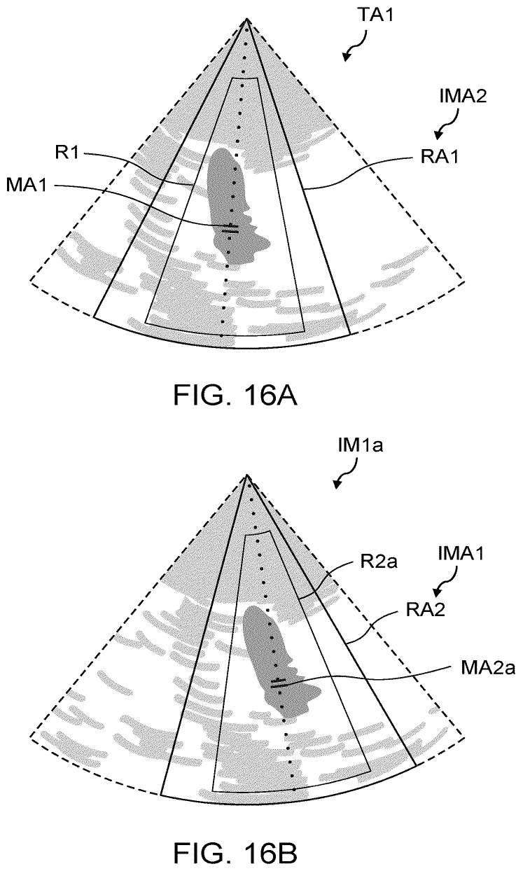

The content of the mode transition processing according to the present embodiment is the same as that of the first embodiment described above. FIGS. 15 and 16 are diagrams for describing steps of step S11 and step S13 in a case where the ultrasonic diagnostic apparatus 1 according to the present embodiment executes the mode transition processing of FIG. 4 described above.

FIG. 15 is a diagram illustrating an example of a step in which the acquisition function 383 acquires a two-dimensional B-mode image and a marker, which is an example of the second identification information, in step S11 of the mode transition processing executed in the ultrasonic diagnostic apparatus 1 according to the eighth embodiment. As illustrated in FIG. 15, in step S11, the acquisition function 383 acquires a two-dimensional B-mode image IM1a and a marker MA2a which is an example of the second identification information. The two-dimensional B-mode image IM1a illustrated in FIG. 15 is an image including a part of the target image TA1 illustrated in FIG. 2.

FIG. 16 is a diagram illustrating an example of a step in which the calculation function 384c calculates a matching degree between the two-dimensional ultrasonic image and the measurement position thereof, and the target image TA1 and the measurement position thereof, in step S13 of the mode transition processing executed in the ultrasonic diagnostic apparatus 1 according to the eighth embodiment. First, the calculation function 384c extracts an image around the marker MA1 in the target image TA1. Specifically, as illustrated in FIG. 16A, the calculation function 384c extracts a second image IMA2 in a predetermined range RA1 centered on the marker MA1 in the target image TA1 in which the measurement position is set by setting the marker MA1. Note that, in the example illustrated in FIG. 16A, the second image IMA2 in the predetermined range RA1 centered on the marker MA1 in the target image TA1 is extracted, but the second image IMA2 may not be the predetermined range RA1 centered on the marker MA2. That is, a method of extracting the second image IMA2 is arbitrary, and the second image IMA2 may be extracted in a predetermined range including the marker MA1 in the target image TA1.

Next, as illustrated in FIG. 16B, the calculation function 384c extracts an image around the marker MA2 in the two-dimensional B-mode image IM1. Specifically, as illustrated in FIG. 16B, the calculation function 384c extracts the first image IMA1 in a predetermined range RA2 centered on the marker MA2 in the two-dimensional B-mode image IM1 in which the measurement position is set by setting the marker MA2. Note that, in the example illustrated in FIG. 16B, the first image IMA1 in the predetermined range RA2 centered on the marker MA2 in the two-dimensional B-mode image IM1 is extracted, but the first image IMA1 may not be the predetermined range RA2 centered on the marker MA1. That is, a method of extracting the first image IMA1 is arbitrary, and the first image IMA1 may be extracted in a predetermined range including the marker MA2 in the two-dimensional B-mode image IM1.

Then, in step S13, the calculation function 384c calculates the matching degree between the first image IMA1 and the second image IMA2 as the matching degree between the two-dimensional ultrasonic image and the first measurement position information, and the target image TA1 and the second measurement position information. Note that, as described above, the processing of steps S15 and S17 after step S13 is the same as that of the first embodiment, and thus the description thereof will be omitted.

As described above, according to the ultrasonic diagnostic apparatus 1 according to the present embodiment, as the matching degree between the two-dimensional ultrasonic image and the first measurement position information, and the target image TA1 and the second measurement position information, the matching degree between the image around the first measurement position information in the two-dimensional ultrasonic image in which the measurement position is set by setting the first measurement position information and the image around the second measurement position information in the target image TA1 in which the measurement position is set by setting the second measurement position information is calculated. Therefore, even if the matching degree between the entire target image TA1 and the entire two-dimensional ultrasonic image is low, in a case where the matching degree between the image around the first measurement position information in the two-dimensional ultrasonic image and the image around the second measurement position information in the target image TA1 is equal to or greater than the threshold value, the transition to the Doppler mode is controlled. Therefore, the user only needs to visualize an image including the image around the second measurement position information in the target image TA1 as the two-dimensional ultrasonic image, and the convenience of the user can be improved.

### OTHER MODIFICATIONS

In the first to seventh embodiments described above, the calculation functions 384, 384a, and 384b may perform weighting on the image according to the measurement position when the matching degree between the images is calculated. Specifically, the calculation functions 384, 384a, and 384b may increase a weighting value of an image in a region close to a position at which a marker indicating the measurement position is set, may set the weighting to the image such that the weighting value decreases as a distance from the position at which the marker indicating the measurement position is set increases, and may calculate a matching degree between the images in consideration of the weighting.

Furthermore, in the first to seventh embodiments described above, a case in which the two-dimensional ultrasonic image and the target image TA1 are the two-dimensional B-mode image IM1 has been described as an example, but the two-dimensional ultrasonic image and the target image TA1 may be two-dimensional color Doppler images. In addition, although a case in which the Doppler mode is the PWD mode has been described as an example, the Doppler mode may be the CWD mode.

Furthermore, in the first to seventh embodiments described above, the first measurement position information and the second measurement position information are markers indicating measurement positions set in the two-dimensional ultrasonic image and the target image TA1, respectively, but the present invention is not limited thereto. That is, the contents of the first measurement position information and the second measurement position information are arbitrary, and for example, may be XY coordinates indicating the position of the marker set in each of the two-dimensional ultrasonic image and the target image TA1 with an arbitrary position on the image as a reference, or may be XY coordinates indicating the position of the marker set in each of the two-dimensional ultrasonic image and the target image TA1 with the ultrasonic probe 10 as a reference.

Furthermore, in the seventh embodiment described above, a case in which the transition control function 385a controls the transition from the PWD mode to the mode for generating the two-dimensional ultrasonic image in a case where the difference between the position of the ultrasonic probe 10 at the time of transitioning to the PWD mode and the current position of the ultrasonic probe 10 exceeds the threshold value or in a case where the blood flow signal cannot be detected has been described. However, the transition control function 385a may control the transition from the PWD mode to another mode different from the mode for generating the two-dimensional ultrasonic image.

Note that the word “processor” used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the memory 37. Note that programs may be configured to be directly integrated in the processor instead of being storing in the memory 37. In this case, the processor realizes functions by reading and executing programs stored in the circuitry. Note that the processor is not limited to the case arranged as a single processor circuit, but may be configured as a single processor by combining a plurality of independent circuits to realize functions. Furthermore, a plurality of component elements in FIG. 1 may be integrated into one processor to realize the functions.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. An ultrasonic diagnostic apparatus comprising:
processing circuitry configured to:
acquire a two-dimensional ultrasonic image generated based on a signal received by an ultrasonic probe and first measurement position information related to a measurement position for measurement of blood flow information, the measurement position being set in the two-dimensional ultrasonic image;
calculate a matching degree between the two-dimensional ultrasonic image and the first measurement position information, and a reference image having a measurement position set therein and second measurement position information related to the measurement position set in the reference image; and
control, based on a calculation result, a transition to a Doppler mode for the measurement of the blood flow information in a subject.

2. The ultrasonic diagnostic apparatus of claim 1, wherein the reference image is any one of an ultrasonic image in which the measurement position is set in a frozen state of live display of the two-dimensional ultrasonic image, an ultrasonic image in which the measurement position is set in a past ultrasonic inspection, and an ultrasonic image in which the measurement position immediately before the transition to the Doppler mode is set.

3. The ultrasonic diagnostic apparatus of claim 1, wherein the processing circuitry is further configured to control a position of a marker indicating the measurement position set on the two-dimensional ultrasonic image so as to increase the matching degree between the first measurement position information and the second measurement position information.

4. The ultrasonic diagnostic apparatus of claim 1, wherein the processing circuitry is further configured to notify a user of the matching degree between the two-dimensional ultrasonic image and the first measurement position information, and the reference image having the measurement position set therein and the second measurement position information related to the measurement position set in the reference image.

5. The ultrasonic diagnostic apparatus of claim 4, wherein the processing circuitry is further configured to notify the matching degree between the two-dimensional ultrasonic image and the first measurement position information, and the reference image and the second measurement position information by one of color, number, and sound, or a combination of two or more of the color, the number, and the sound.

6. The ultrasonic diagnostic apparatus of claim 1 comprising: a position sensor configured to detect a position of the ultrasonic probe.

7. The ultrasonic diagnostic apparatus of claim 6, wherein the processing circuitry is further configured to calculate, in addition to the matching degree between the two-dimensional ultrasonic image and the first measurement position information, and the reference image having the measurement position set therein and the second measurement position information related to the measurement position set in the reference image, a matching degree between a position of the ultrasonic probe when the reference image is generated, the position being detected by the position sensor, and a current position of the ultrasonic probe, the current position being detected by the position sensor.

8. The ultrasonic diagnostic apparatus of claim 6, wherein the processing circuitry is further configured to:
determine whether a difference between a position of the ultrasonic probe when the transition to the Doppler mode is performed, the position being detected by the position sensor, and a current position of the ultrasonic probe, the current position being detected by the position sensor, exceeds a predetermined threshold value; and
control, in a case where the difference exceeds the predetermined threshold value, a transition from the Doppler mode to a mode for generation of the two-dimensional ultrasonic image.

9. The ultrasonic diagnostic apparatus of claim 1, wherein the processing circuitry is further configured to control, in a case where a blood flow signal is not detectable in the Doppler mode, a transition from the Doppler mode to a mode for generation of the two-dimensional ultrasonic image.

10. The ultrasonic diagnostic apparatus of claim 1, wherein the processing circuitry is further configured to control, at a time of the transition to the Doppler mode, a display unit such that a live display of the two-dimensional ultrasonic image is frozen and a Doppler waveform in the blood flow information is displayed live.

11. The ultrasonic diagnostic apparatus of claim 10, wherein the processing circuitry is further configured to control the display unit so as to display, in addition to the two-dimensional ultrasonic image and the blood flow information, at least one of the reference image and a body mark related to the two-dimensional ultrasonic image.

12. The ultrasonic diagnostic apparatus of claim 10, wherein the processing circuitry is further configured to cause the display unit to display at least one of the reference image and a body mark side by side together with the two-dimensional ultrasonic image and the blood flow information.

13. The ultrasonic diagnostic apparatus of claim 10, wherein the processing circuitry is further configured to cause the display unit to display at least one of the reference image and a body mark related to the two-dimensional ultrasonic image in a superimposed manner on at least one of the two-dimensional ultrasonic image and the blood flow information.

14. The ultrasonic diagnostic apparatus of claim 1, wherein the processing circuitry is further configured to calculate, as the matching degree between the two-dimensional ultrasonic image and the first measurement position information, and the reference image and the second measurement position information, a matching degree between an image around the first measurement position information in the two-dimensional ultrasonic image in which the measurement position is set by setting the first measurement position information and an image around the second measurement position information in the reference image in which the measurement position is set by setting the second measurement position information.
